(19) United States
(12) Patent Application Publication
(10) Pub. No.: US 2025/0387556 A1
(43) Pub. Date: Dec. 25, 2025

(54) VASCULAR DEVICE LOADING

(71) Applicant: MicroVention, Inc., Aliso Viejo, CA (US)

(72) Inventors: Joseph Gulachenski, Trabuco Canyon, CA (US); Roland Guyon, Santa Ana, CA (US); Jessica Chin, Aliso Viejo, CA (US)

(21) Appl. No.: 18/879,572

(22) PCT Filed: Jun. 29, 2023
(86) PCT No.: PCT/US2023/069337

§ 371 Date: Dec. 27, 2024

Related U.S. Application Data
(60) Provisional application No. 63/356,970, filed on Jun. 29, 2022.

(51) Int. Cl. A61M 5/14; A61M 5/00; A61M 5/145; A61M 25/00; A61M 39/02

(52) U.S. Cl. CPC A61M 5/1409; A61M 5/007; A61M 5/1452; A61M 25/0097; A61M 39/0247; A61M 2005/1402; A61M 2039/0205; A61M 2039/0258

(57) ABSTRACT

Vascular device loading may be provided by loading a first liquid having a first density into a vascular device hub having an internal volume flowably connected between a syringe port and a cannula port; connecting a first syringe loaded with a second liquid to the syringe port, wherein the second liquid has a second density different than the first density; orienting the first syringe at a first elevation relative to the vascular device hub to place a denser one of the first liquid and the second liquid below a less dense one of the first liquid and the second liquid; and injecting, from the first syringe into the vascular device hub, the second liquid to eject the first liquid from the vascular device hub via the cannula port.

### CROSS-REFERENCES TO RELATED APPLICATIONS

The present disclosure claims the benefit of U.S. Provisional Patent Application No. 63/356,970 entitled “PROCESSES FOR LOADING VASCULAR DEVICES” and filed on 2022 Jun. 29, which is incorporated herein by reference in its entirety.

### FIELD

The present disclosure generally relates to the use of devices for delivering fluids into the vasculature of a biological subject. More particularly, the present disclosure described an improved process or method for delivering, via a single catheter and syringe assembly, multiple fluids while avoiding or reducing mixing between those fluids, for the improved treatment of various medical conditions in the biological subject.

### SUMMARY

The present disclosure provides vascular device loading, such as for the treatment or prophylaxis of medical conditions by the targeted delivery of multiple separate fluids. For example, during a liquid embolic procedure, five flushes of liquids may be used (e.g., a first saline injection to prepare a catheter, a contrast agent injection to confirm catheter location and visualize the vasculature of the biological subject, a second saline injection to flush out the contrast, a DMSO injection to prepare the target location for the injection of a liquid embolic, and a liquid embolic injection to treat an AVM or aneurysm). Because liquid embolic solutions are known to harden prematurely (e.g., not at the target site) when the embolic comes into contact with blood, contrast, saline, or combinations thereof, avoiding the mixing of fluids in the catheter and syringe is important to reduce the risk of premature hardening. Accordingly, proper loading of the vascular devices, as described herein, can improve the delivery of the fluids to the target site, reduce the risk of exerting excessive pressure on the equipment or the biological subject, reduce operator strain or fatigue, reduce the amount of fluids needed to be injected (e.g., via improved on-target delivery), and various other benefits that will be apparent to one of ordinary skill in the art on a detailed reading.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 shows a syringe oriented about 60 degrees from vertical with a tip thereof pointed upward, connected to a vascular device hub, the syringe including a second liquid that is denser than a first liquid in the vascular device hub, according to embodiments of the present disclosure.

FIG. 2 shows a syringe oriented substantially vertically with a tip thereof pointed upward, connected to a vascular device hub, the syringe including a second liquid that is denser than a first liquid in the vascular device hub, according to embodiments of the present disclosure.

FIG. 3 shows avoidance of introducing an air bubble into a vascular device system, the syringe including a second liquid that is denser than a first liquid in the vascular device hub, according to embodiments of the present disclosure.

FIG. 4 shows undesired channeling or back-flow of two liquids through one another, the syringe including a second liquid that is denser than a first liquid in the vascular device hub, according to embodiments of the present disclosure.

FIG. 5 shows undesired channeling or back-flow of two liquids through one another in a vascular device containing a first liquid in the vascular device hub where the first liquid is less dense than a second liquid in the syringe, according to embodiments of the present disclosure.

FIG. 6 shows a syringe oriented about 30 degrees from vertical with a tip thereof pointed downward, connected to a vascular device hub, the syringe including a second liquid that is less dense than a first liquid in the vascular device hub, according to embodiments of the present disclosure.

FIG. 7 shows undesired channeling or bubbling of a second liquid in a syringe through a first liquid in a vascular device hub, where the first liquid is denser than the second liquid in the syringe, according to embodiments of the present disclosure.

FIG. 8 shows a syringe substantially vertically oriented with a tip thereof pointed downward and connected to a vascular device hub, where the syringe includes a second liquid that is denser than a first liquid in the vascular device, and the device hub includes a third liquid flowably connected to the first and second liquid, the third liquid denser than the first liquid and second liquid, according to embodiments of the present disclosure.

FIG. 9 is a flowchart of an example method for vascular device loading, according to embodiments of the present disclosure.

FIGS. 10A-10C are flowcharts of example methods of use, according to embodiments of the present disclosure.

As will be appreciated, the various fluid levels and devices have been simplified for purposes of explanation and so as to not distract from the features discussed in the present disclosure.

### DETAILED DESCRIPTION

Various embolics are available for addressing interruptions to liquid flow paths in a lumen or vasculature, and include liquid or non-liquid materials. Numerous disease states, including but not limited to arteriovenous malformations (AVMs) or aneurysms may be treated by filling with one or more embolics. Some aneurysm treatment procedures include use of a catheter (e.g., a microcatheter) and a series of liquid volumes pushed through the catheter, including contrast liquids, flush liquids, pre-load liquids, and embolic liquids, and combinations thereof. Some liquid embolic polymer may be designed to coagulate, precipitate, or otherwise release itself from a flowable organic solution at the interface of a contact with an aqueous liquid. Subsequent leaching or diffusion of the organic solvent from the liquid embolic allows the embolic polymer to further solidify.

Such treatment procedures present with a variety of risks, including fouling of a device used to deliver an embolic by, for example, premature lodging, coagulation, or precipitation of the embolic in the device or components used to deliver the embolic to the device. It has been discovered that density differences of liquids serially flowed through vascular devices require one or more particular features of a process to reduce the risk of a fouled device when preparing to administer, or when administering, an occlusive material such as a liquid embolic through the device.

Thus, provided herein are methods or processes of loading a vascular device with an embolic. Also provided herein are methods of reducing the risk of premature liquid embolic solidification or unfavorable flow characteristics resulting in catheter rupture or embolic behavior while using a vascular device (e.g., a microcatheter) to treat an aneurysm in a biological subject in need thereof. Also provided herein are methods of treatment or prophylaxis of AVMs or aneurysms in a biological subject

In some embodiments, the process features referred to above include a syringe's maximum angle from vertical to prevent settling of one liquid into another or to prevent channeling of one liquid through or around another, the density or viscosity of a liquid flushing agent, the density or viscosity of a liquid residing in a lumen of a vascular device, or a combination thereof.

As discussed herein, the present disclosure makes reference to the angle and position of various components. Unless explicitly stated otherwise, the orientation of these components and elements is in reference to the longitudinal axis of that component or element in relation to the environment in which the component or element is found. As used herein, the vertical upward position corresponds to 0 degrees of rotation, the horizontal position corresponds to 90 degrees of rotation from the vertical upward position, and the vertical downward position corresponds to 180 degrees from the vertical upward position (and 90 degrees from the horizon towards the floor). For example, a hypothetical fluid would settle to have a surface (ignoring the effects of a meniscus) that is horizontal in the reference frame. A float positioned in this hypothetical fluid would have a portion located vertically upward relative to the fluid (e.g., that floats above the fluid surface in the reference frame) and portion located downward relative to the fluid (e.g., that is submerged below the fluid surface in the reference frame).

In some embodiments, when loading a vascular device with a liquid polymer embolic that will release from a flowable organic solution on contact with an aqueous liquid, it is important to flush all or substantially all aqueous liquid that may reside in the vascular device (e.g., due to prior pre-loading steps) before the embolic is loaded into the vascular device. Doing so reduces the risk of premature release of the polymeric embolic out of solution within the vascular device, which would require increased force to cause the liquid embolic to flow from a syringe through the vascular device to a target in a biological subject and increased stress on the biological subject.

In some embodiments, the processes provided herein includes loading a vascular device with a liquid embolic, and may further include a pre-loading processes described herein.

In some embodiments, a process for pre-loading a vascular device may include 1) pushing a volume of liquid contrast through the device at a particular flow rate, 2) pushing a volume of normal saline through the device at a particular flow rate, 3) pushing a volume of flush liquid through the device at a particular flow rate to pre-load the device for loading with a liquid embolic. Each of the liquid contrast, saline, flush liquid, and liquid embolic may have different densities or viscosities, or both. Furthermore, in some embodiments, each of the liquid contrast, saline, flush liquid, and liquid embolic may have different base carrier solvents. That is, in some embodiments, the liquid embolic may be in an organic solvent, such as dimethyl sulfoxide (DMSO) or the like, whereas saline is an aqueous solution of a salt and water. Similarly, in some embodiments, the flush liquid may be in an organic solvent, such as DMSO or the like. Additionally, in some embodiments, the liquid contrast may be aqueous based or in an organic carrier, or a combination thereof.

Vascular device pre-loading sequences include one or more steps where a second liquid volume is pushed through the vascular device containing a first liquid volume. In some embodiments, each of the liquid contrast, normal saline, flush liquid, and liquid embolic may be, independently, the first liquid volume or the second liquid volume.

In some embodiments, when the first liquid volume is less dense than the second liquid volume and the first liquid volume and second liquid volume are to be flowably connected, a syringe including the second liquid volume is operably connected to the vascular device and oriented between about 60 degrees from vertical to substantially vertical, with the syringe tip pointed up when pushing the second liquid volume through the vascular device.

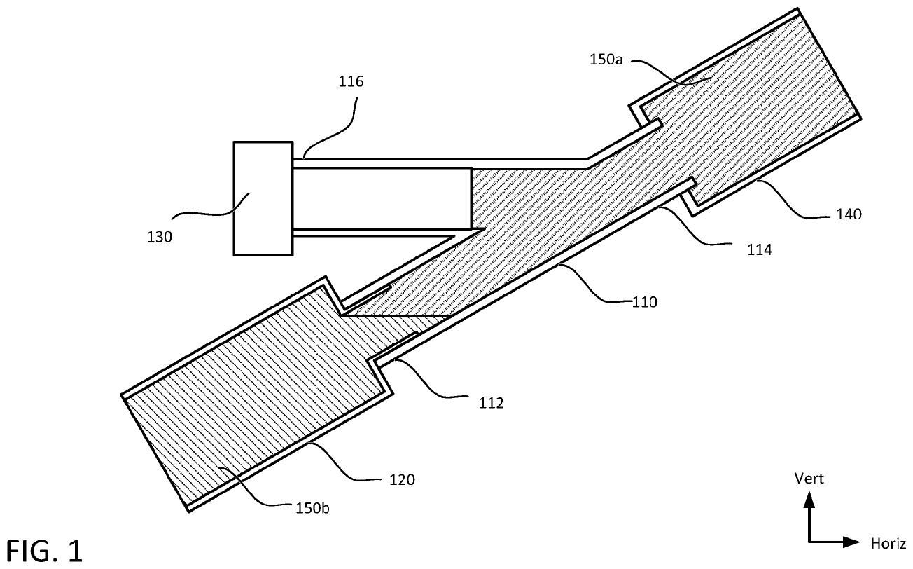

FIGS. 1-8 show various interactions between different fluids, according to various embodiments of the present disclosure. Each of FIGS. 1-8 illustrate a portion of a syringe 120, connected used to deliver a liquid to a vascular hub device 110. The vascular hub device 110 includes a syringe port 112 provided to establish fluid communication with the syringe 120. On another end of the vascular hub device 110 to the first syringe port 112a is a cannula port 114 to which a first end of a catheter 140 is shown attached. An opposite end of the catheter 140 (not shown) is inserted into a biological subject to deliver various fluids or devices to a target location in the biological subject (e.g., a site of an AVM or aneurysm). In some embodiments, the vascular hub device 110 also includes a device port 116, though which a plug 130 is shown that blocks fluid communication between the inner volume of the vascular device hub and the external environment. The plug 130 allows selective access for various tools, dilators, guidelines or other devices to the biological subject, or the application of an additional syringe, negative pressure source, or the like.

In each of FIGS. 1-8, various liquids 150a-c are shown (generally or collectively, liquids 150). As described in greater detail in regard to the individual FIGS. 1-8, the liquids 150 may be separated based on individual densities, may mix, or percolate through one another to establish equilibrium according a density flow through a shared space.

In various embodiments, the catheter 140 through which the aforementioned liquids 150 flow may be coated or uncoated. In some embodiments, an interior surface of the catheter 140 may be hydrophobic, hydrophilic, or amphiphilic.

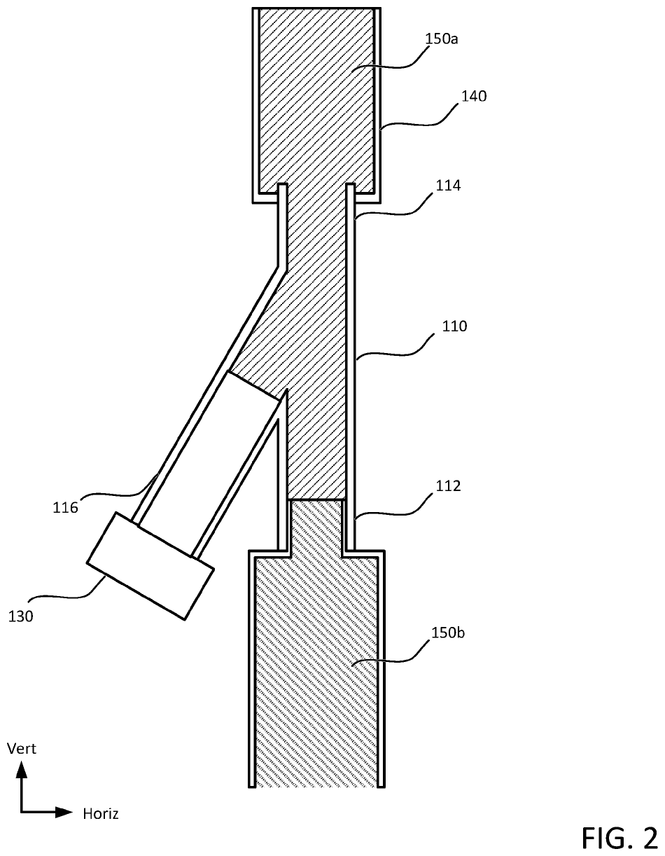

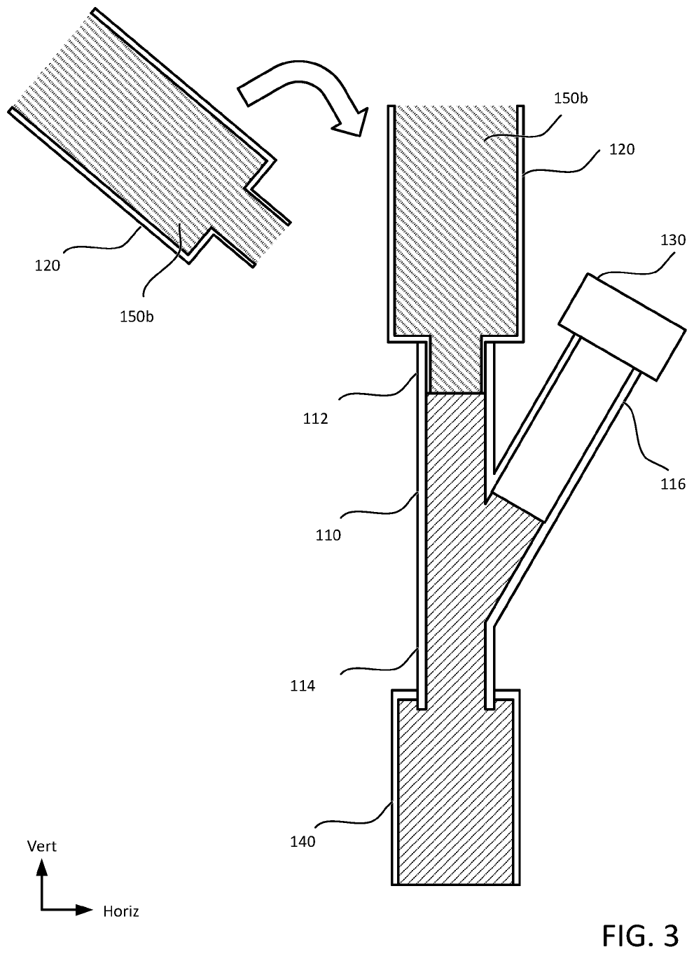

Some examples of density separation are shown in FIGS. 1 and 2 where a first liquid 150a of saline is present in the vascular device hub 110 and catheter 140, and a second liquid 150b of a DMSO solution is present in the syringe 120. In some embodiments, to avoid introduction of an air bubble to the liquids 150 being pushed through the vascular device 110, the syringe 120 including the second liquid 150b has its tip oriented downward when connecting to the vascular device hub 110 (for example, as shown in FIG. 3), and the syringe 120 is then oriented to be substantially vertical (as in FIG. 2). In some embodiments, the syringe 120 is substantially vertical when connected to the syringe port 112 when the longitudinal axis of the syringe 120 is are not more than 45 degrees from vertical, more preferably not more than 30 degrees from vertical, and even more preferably not more than 15 degrees from vertical. In some embodiments, such a sequence of tipping between vertically upward and vertically downward reduces an undesired mixing of the two liquids 150 when the liquids 150 have different densities when in the final state the denser liquid 150 is below the less dense liquid 150.

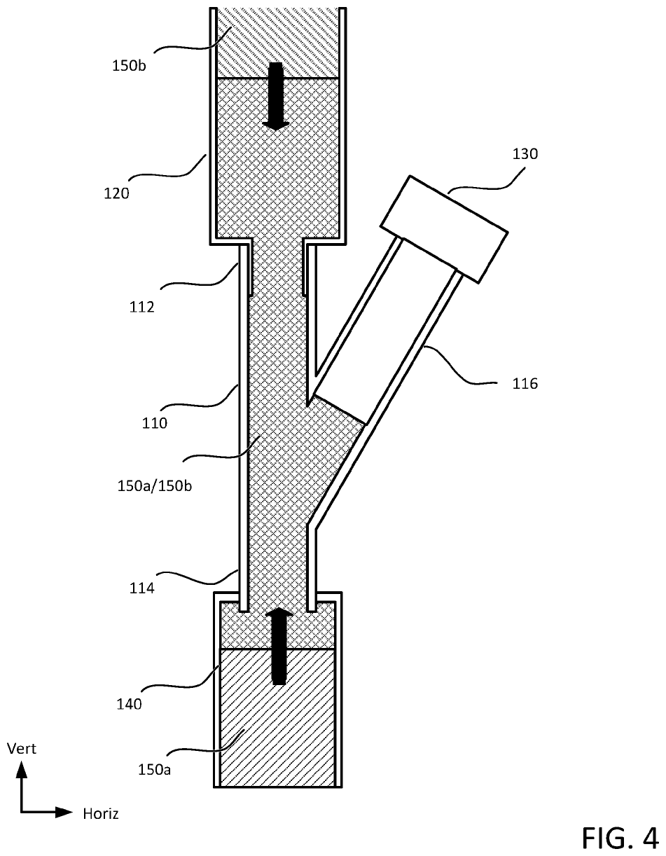

FIGS. 4 illustrate examples of undesired channeling of two immiscible liquids 150 with different densities, which the present disclosure seeks to mitigate. For example, by leaving the syringe 120 with the more dense second liquid 150b above the less dense first liquid 150a for too long, the two liquids 150 can channel through one another so that the less dense liquid 150 forms a layer on top of the more dense liquid 150.

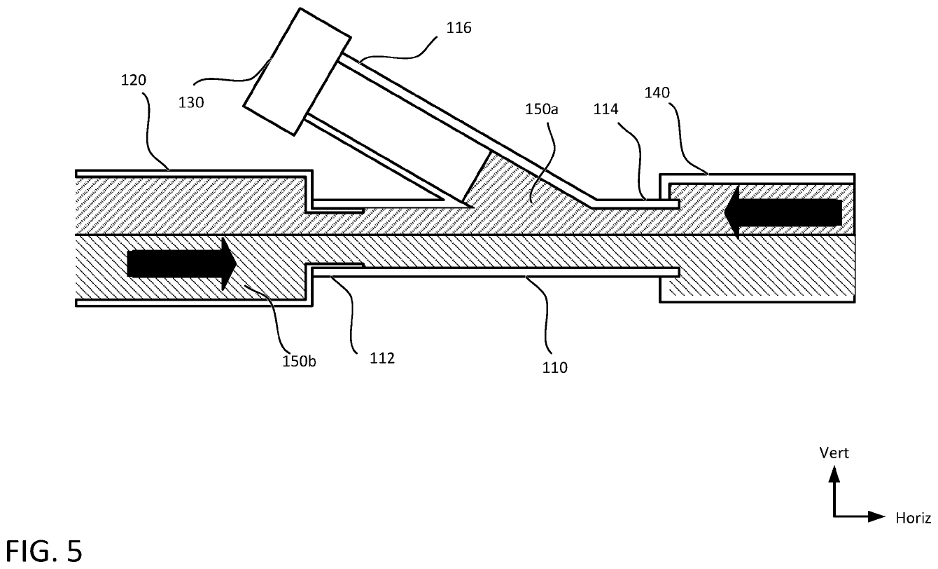

Another undesirable channeling of two liquids may occur as shown in FIG. 5, in which the second liquid 150b is injected through the vascular device hub 110, but due to the horizontal orientation of the devices, results in back-flow of the first liquid 150a from a vascular device hub 110 rather than injection through the catheter 140.

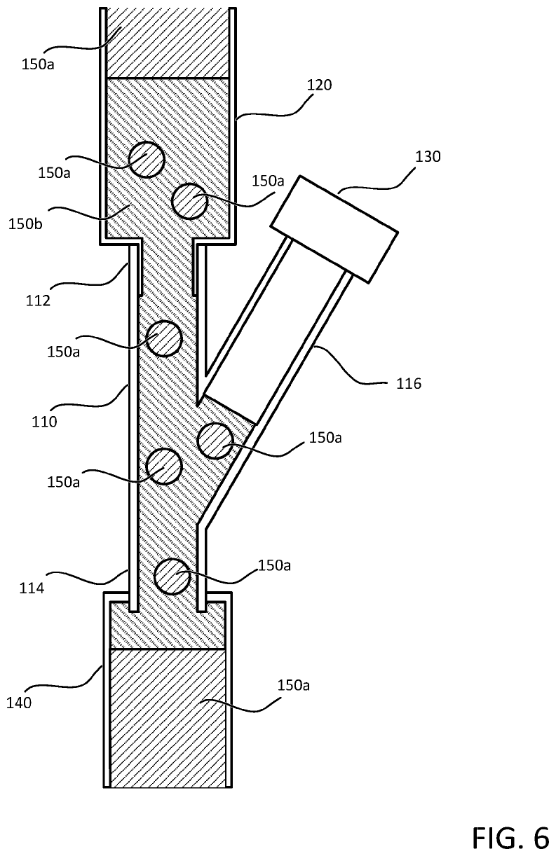

Another undesirable channeling of two liquids may occur as shown in FIG. 6, which shows undesirable channeling or bubbling of a first liquid 150a (e.g., saline) through a denser second liquid 150b (e.g., a contrast agent) through the a vascular device hub 110 when the syringe 120 is held at a substantially vertical orientation while pushing the first liquid 150a from the syringe 120 into the vascular device hub 110.

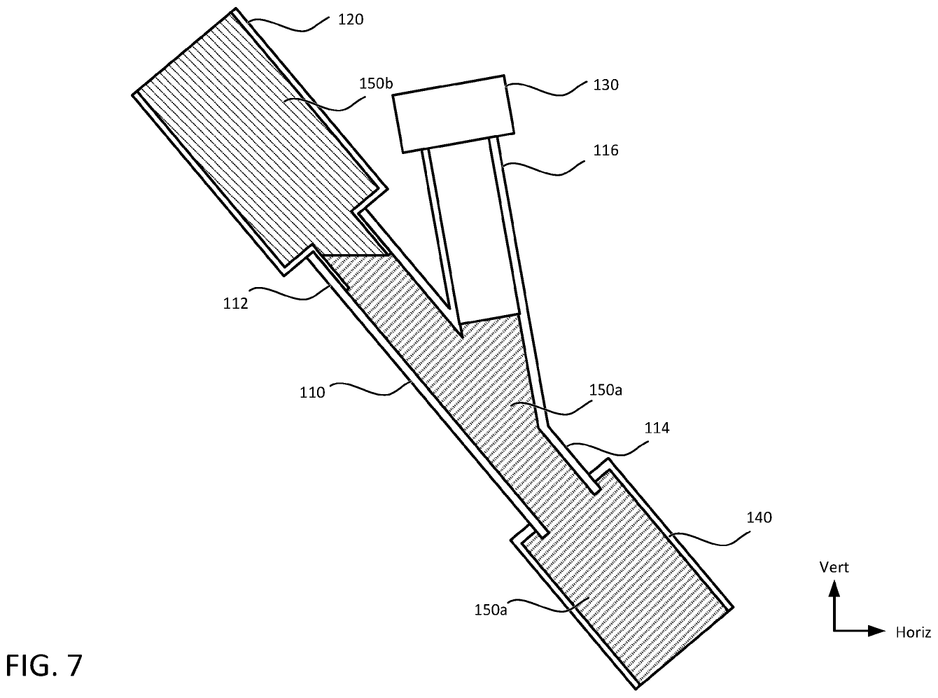

FIG. 7 shows an orientation of the syringe 120 relative to the vascular device hub 110 where a syringe 120 including the second liquid 150b is operably connected to the vascular device hub 110. As illustrated, the syringe 120 is oriented between about 30 degrees from vertical, but may be oriented at other angles between 45 degrees to substantially vertical, with the syringe tip pointed downward. In the illustrated example, because the first liquid 150a is denser than the second liquid 150b, when the first liquid 150a and second liquid 150b are placed in fluid communication (e.g., are flowably connected), the second liquid 150b floats on the first liquid 150a, and does not channel or mix with the first liquid 150a, in contrast to the behavior of the opposing orientation or a horizontal orientation as discussed in relation to FIGS. 4-6.

In some embodiments, the liquid embolic has a viscosity of about 12 centipoise or more. In some embodiments, the liquid embolic is denser than a typical saline solution. In some embodiments, the liquid contrast is more dense than a typical saline solutions. In some embodiments, the liquid embolic is denser than the liquid contrast. Accordingly, when sequentially loading and injection of various fluid volumes of saline solutions, liquid embolics, liquid contrasts, etc. with different densities, care should be taken for the orientation of the syringe 120 introducing a new liquid 150 relative to the vascular device hub 110 holding an earlier-introduced liquid 150.

For example, to prevent channeling of a DMSO based second liquid 150b in a syringe 120, through a first liquid 150a in a vascular device hub 110 consisting of a saline solution or to prevent back-flow of the first liquid 150a into the syringe 120, a bolus of a third liquid 150c (such as a liquid contrast agent) may be flowably located between the interface between the first liquid 150a and the second liquid 150b. In various embodiments, the volume of the bolus will vary based on the volume of the vascular device hub 110, but in some embodiments, the volume of the bolus is up to about 500 microliters (μL).

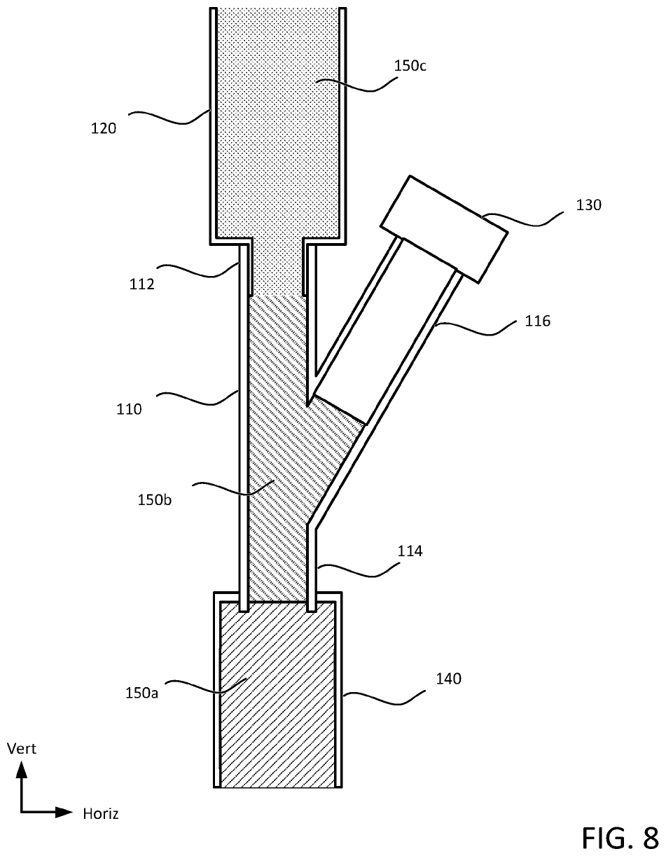

FIG. 8, shows such a bolus of the third liquid 150c, which may be obtained by first loading the vascular device hub 110 with the first liquid 150a and then loading the third liquid 150c into the vascular device hub 110 with a syringe 120 pointed upward (as in FIG. 2). For example, enough of the third liquid 150c is injected to fill the vascular device hub 110 (e.g., 0.2 milliliters (mL)), and the catheter 140 still contains the first liquid 150a. Once the bolus is loaded into the vascular device hub 110, an operator may remove the syringe 120 that carried the third liquid 150c, and attach another syringe 120 that carries the second liquid 150b to the syringe port 112 with the new syringe 120 pointed downward (as in FIG. 8). Accordingly, using the different orientations for introduction of the various liquids 150, there is a decreased chance of mixing or channeling to occur. It is noted that saline is less dense than DMSO, which is less dense than contrast liquid.

One challenge in understanding the fluid mechanics of how these various liquids 150 flow through the system is visualization. Many of the liquids 150 have similar or identical colors, or are difficult to visually distinguish through opaque, colored, or semi-transparent components (catheters, microcatheters, syringes, etc.). Creating “real-world” conditions in a laboratory setting may require making transparent versions of products that are typically purchased “off the shelf” as opaque or colored. Once transparent components are created, then colored dyes or other distinguishing characteristics may be added to the various liquids 150 to distinguish the liquids 150 from one another, which may require testing to confirm that the addition of dyes or other factors did not modify or alter the relevant chemical or fluid properties. Once the chemical and fluid properties are confirmed, then the modified fluids can be used with the modified (transparent) components to visualize the fluid mechanics within the implantable system in real time, and various angles, flushing techniques, and orders of operations can be tested.

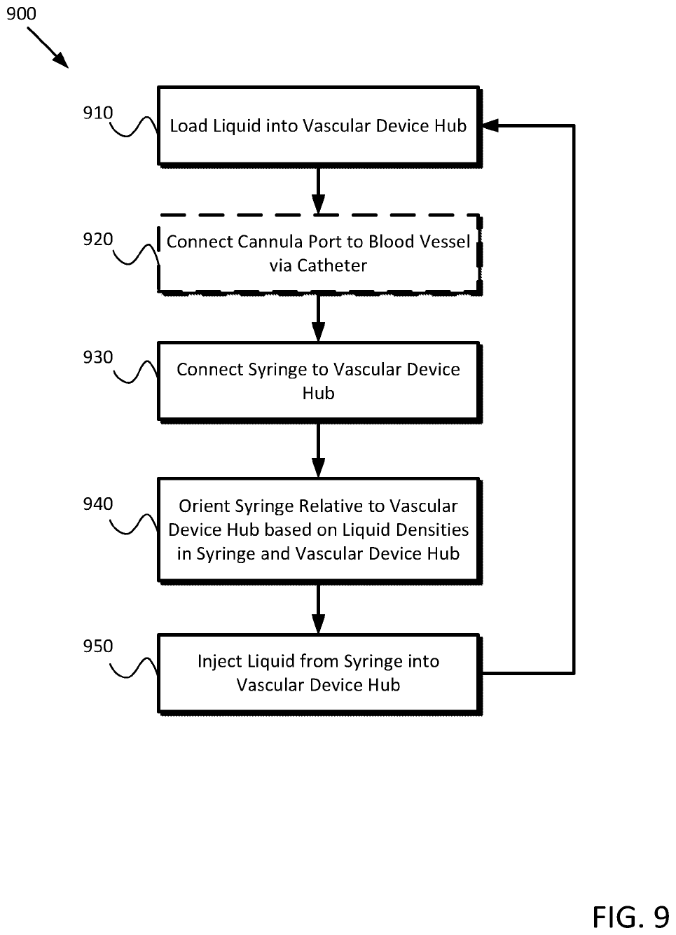

FIG. 9 is a flowchart of an example method 900 for vascular device loading, according to embodiments of the present disclosure. Method 900 may repeat across several iterations of block 910-950 to load various liquids 150 in sequence through a vascular device used for the delivery of liquids 150 to a target site in a biological subject. Accordingly, although the examples given herein recite three liquids 150a-c, the present disclosure contemplates that two liquids 150a-b or more than three liquids 150 may also be loaded according to method 900.

For example, during a liquid embolic procedure, five flushes of liquids may be used (e.g., a first saline injection to prepare a catheter, a contrast agent injection to confirm catheter location and visualize the vasculature of the biological subject, a second saline injection to flush out the contrast, a DMSO injection to prepare the target location for the injection of a liquid embolic, and a liquid embolic injection to treat an AVM or aneurysm). Because liquid embolic solutions are known to harden prematurely (e.g., not at the target site) when the embolic comes into contact with blood, contrast, saline, or combinations thereof, avoiding the mixing of fluids in the catheter and syringe is important to reduce the risk of premature hardening.

Because liquid embolic procedures are often lengthy, and require an operator to manually inject the various liquids to the target site (e.g., via depressing the plunger of a syringe), method 900 prescribes various positions and orientations for the syringe relative to the vascular device hub at certain times to reduce the risk of mixing of fluids of different densities to thereby reduce the risk of (or amount of) the liquid embolic that hardens before delivery to the target site.

At block 910, an operator loads a liquid into the vascular device hub. In various embodiments, the liquid may be loaded via injection from a syringe (e.g., per block 950), suction or backflow from a source, or during manufacture of the vascular device hub.

At block 920, an operator connects the cannula port of the vascular device hub to a blood vessel in a biological subject via a catheter. In various embodiments, the catheter may be inserted into the blood vessel before or after being connected to the cannula port. An operator may also navigate the opposing end of the catheter

In various embodiments, block 920 may be performed before block 910 or may be omitted in an iteration of blocks 910-950. For example, an operator may attach the cannula port to a catheter only once, despite loading multiple fluids into the medical apparatus for injection via the catheter 140 to a target site in a biological subject; the initial connection can be maintained across several loadings and injections of different fluids.

At block 930, the operator connects the syringe to the vascular device hub. In various embodiments, the syringe may directly screw into, snap onto, or be held in place to the vascular device hub with pressure. In some embodiments, tubing may connect between the tip of the syringe and the syringe port; allowing an operator additional ergonomic options for how to hold the syringe in hand while positioning the two fluids at different heights. In various embodiments, various needles, gaskets, or the like may be used to establish a pressure-tight seal for the delivery of a fluid held by the syringe to the vascular device hub. In some embodiments, to avoid introducing air bubbles into the fluid(s) already loaded into the vascular device hub, the syringe is connected to the vascular device hub with a tip of the syringe pointed downward while the syringe is oriented substantially vertically (e.g., as in FIG. 3).

At block 940, the operator orients the syringe relative to the vascular device hub based on the densities of the liquids in the syringe and the vascular hub device, respectively, such that the syringe is at an elevation relative to the vascular device hub to place a denser one of the liquids below a less dense one of the liquids.

For example, with a first liquid that consists of a saline solution (loaded per a first iteration of block 910) and a second liquid in the syringe (connected per a first iteration of block 930) that consists of an aqueous contrast agent solution that is denser than the saline solution, the saline solution (and the vascular device hub) is placed above the aqueous contrast agent solution (and the syringe). Continuing the example, when flushing the contrast with saline (e.g., in a second iteration of block 910-950), the orientation the devices are reversed so that the saline solution (now in the syringe) is placed above the aqueous contrast agent solution (now already loaded in the vascular device hub).

For example, with a first liquid that consists of a DMSO solution (loaded per a current iteration of block 910) and a second liquid in the syringe (connected per a current iteration of block 930) that consists of a liquid embolic, which less dense than the DMSO solution, the DMSO solution (and the vascular device hub) is placed below the liquid embolic (and the syringe).

At block 950, the operator injects the liquid from the syringe into the vascular device hub, which ejects some or all of the liquid previously loaded into the vascular device hub out of the cannula port and towards the biological subject. In various embodiments, depending on the volume of liquid held in the syringe, and the volume of liquid held in the vascular device hub, the injection from the syringe may flush out the vascular device hub; moving at least 50% of the volume of the vascular device hub out through the cannula port. In some embodiments, the injection may loads a bolus of a liquid in the vascular device hub (e.g., as part of a subsequent iteration of block 910) to act as a buffer with a next liquid to be injected (e.g., per a subsequent iteration of blocks 910-950).

Method 900 may repeat through several iterations to provide successive volumes of fluids to a target area of a biological subject to treat an AVM or aneurysm or other condition treatable via targeted delivery of multiple different fluids. Method 900 may be provided in a set of instructions for a medical device (such as a syringe, vascular device hub, catheter, or kit/assembly thereof).

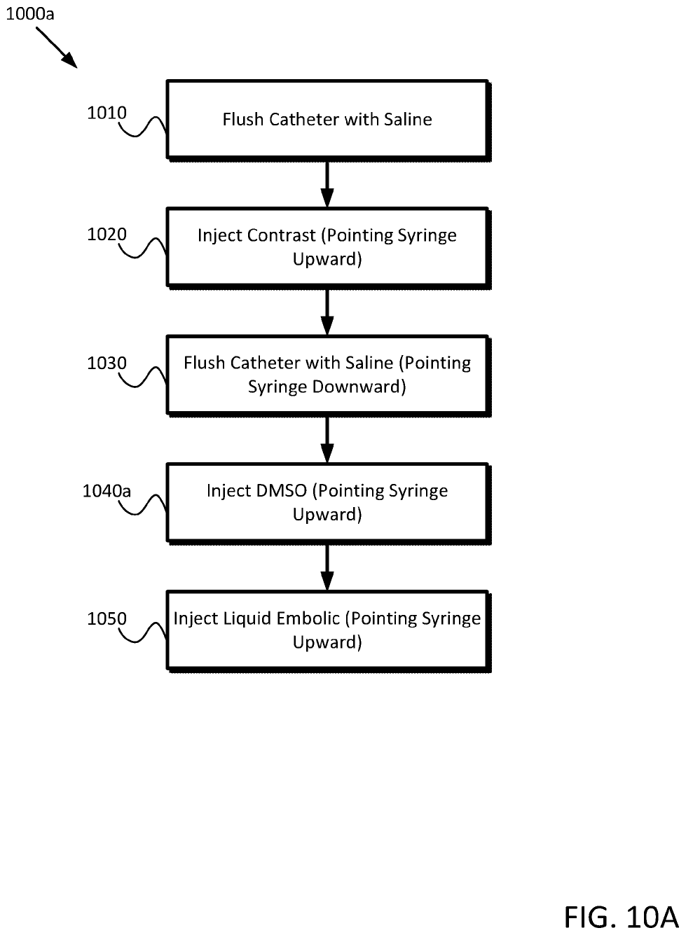

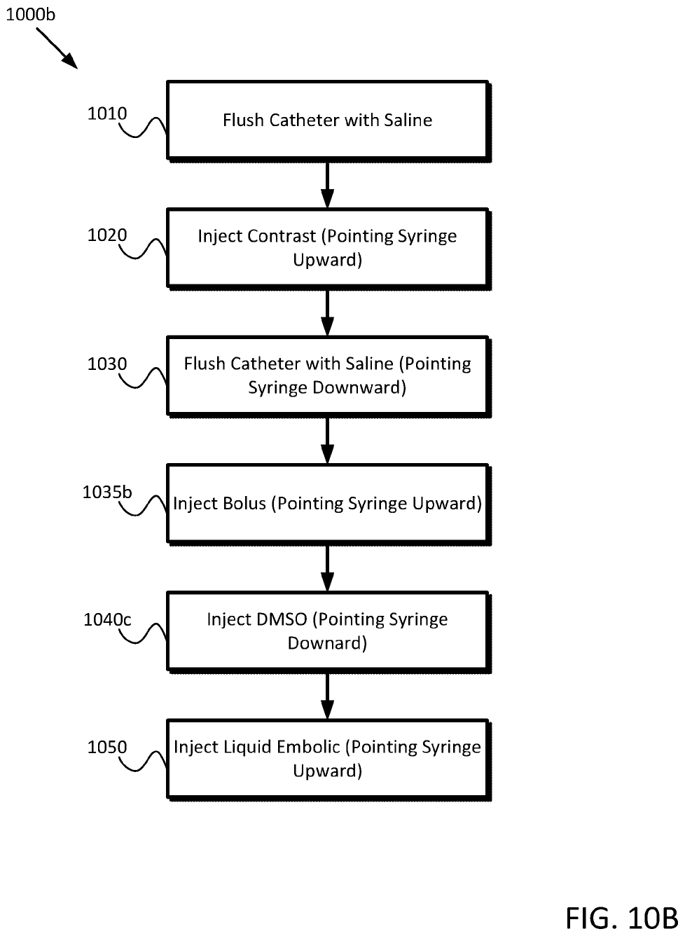

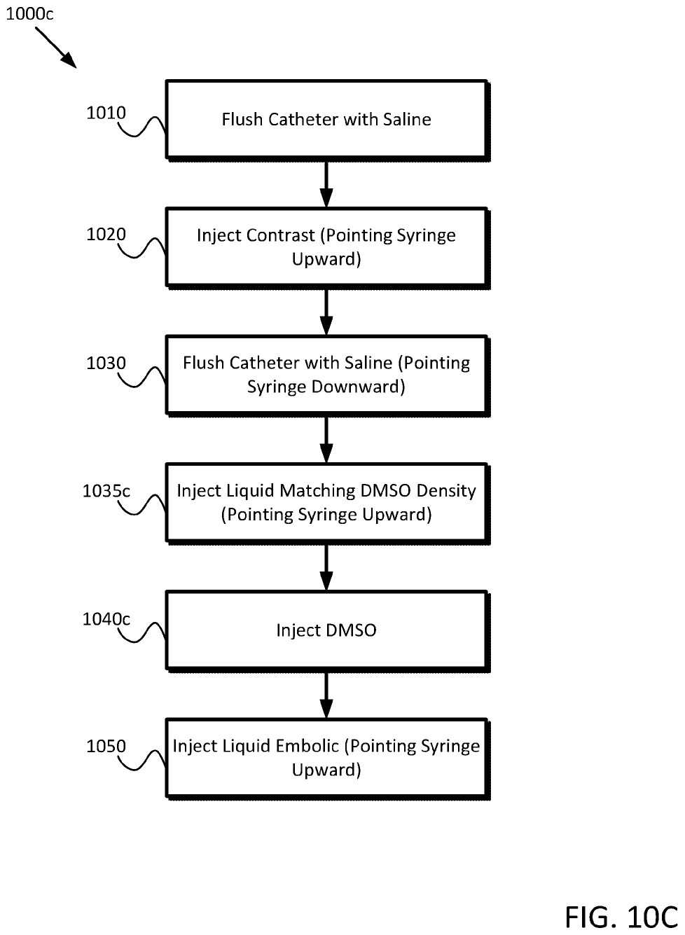

FIGS. 10A-10C are flowcharts of example methods 1000a-c of use, according to embodiments of the present disclosure.

FIG. 10A is a flowchart for a first example method 1000a of use when performing liquid embolic injection for liquid embolics such as cyanoacrylate glues (e.g., Histoacryl (n-butyl cyanoacrylate), Glubran (n-butyl cyanoacrylate plus metacryloxysulpholane, Magic glue or Purefill (n-hexyl cyanoacrylate), TruFill (n-butyl cyanoacrylate), or Fuaile (n-butyl cyanoacrylate plus 2-octyl cyanoacrylate)), Onyx (a liquid embolic system (LES) of a pre-mixed, radiopaque, injectable embolic fluid consisting of the following components: EVOH (ethylene vinyl-alcohol copolymer), DMSO (dimethyl-sulfoxide) and TA (micronized tantalum powder)), Squid (EVOH, DMSO, TA), Menox (EVOH, TA, DMSO), or Precipitating Hydrophobic Injectable Liquid (PHIL; polylactide-co-glycolide, polyhydroxyethyl-methacrylate, triiodophenol, DMSO), in which five liquid flushes are performed.

At block 1010, the operator flushes the catheter with saline to prepare the catheter. In various embodiments, as the catheter is initially empty, the operator may orient the saline-containing syringe orientation in any direction (e.g., pointing upward, downward, horizontally).

At block 1020, the operator injects a contrast (e.g., to confirm catheter location in the vasculature). Because the contrast is denser than saline, when injecting the contrast the operator orients the contrast-containing syringe to point the syringe upward.

At block 1030, the operator flushes the catheter with saline to flush out the contrast. Because saline is less dense than the contrast, the operator orients the saline-containing syringe to point the syringe downward.

At block 1040a, the operator injects DMSO into the catheter in preparation for injecting the liquid embolic (e.g., per block 1050). Because DMSO is denser than the saline injected in block 1030, the operator orients the DMSO-containing syringe orientation to point the syringe upward.

At block 1050, the operator injects the liquid embolic (e.g., Onyx) to perform the procedure on the vasculature of the biological subject into the catheter. Because the liquid embolic is denser than the DMSO injected to block 1040a, the operator orients the embolic-containing syringe to point upward.

FIG. 10B is a flowchart for a second example method 1000b in which a contrast flush (e.g., of about 0.2 mL) is performed to fill the catheter hub and act as a bolus (e.g., a liquid “plug”) before DMSO injection.

At block 1010, the operator flushes the catheter with saline to prepare the catheter. In various embodiments, as the catheter is initially empty, the operator may orient the saline-containing syringe orientation in any direction (e.g., pointing upward, downward, horizontally).

At block 1020, the operator injects a contrast (e.g., to confirm catheter location in the vasculature). Because the contrast is denser than saline, when injecting the contrast the operator orients the contrast-containing syringe to point the syringe upward.

At block 1030, the operator flushes the catheter with saline to flush out the contrast. Because saline is less dense than the contrast, the operator orients the saline-containing syringe to point the syringe downward.

At block 1035b, the operator fills the catheter with a bolus or “plug” of liquid to separate the saline injected in block 1030 and the DMSO that will be injected in block 1040b. Because the liquid used for the bolus is denser than saline (e.g., a contrast agent like that used in block 1020), the operator orients the bolus liquid-containing syringe to point the syringe upward.

At block 1040b, the operator injects DMSO into the catheter in preparation for injecting the liquid embolic (e.g., per block 1050). Because DMSO is separated from the saline injected in block 1030 by the bolus injected per block 1035b, which may be a denser liquid than the DMSO, the operator orients the DMSO-containing syringe orientation to point the syringe downward.

At block 1050, the operator injects the liquid embolic (e.g., Onyx) to perform the procedure on the vasculature of the biological subject into the catheter. Because the liquid embolic is denser than the DMSO injected to block 1040b, the operator orients the embolic-containing syringe to point upward.

FIG. 10C is a flowchart for a third example method 1000c in which an additional pharmaceutically acceptable liquid that matches the density of DMSO is used as a saline purge before DMSO injection.

At block 1010, the operator flushes the catheter with saline to prepare the catheter. In various embodiments, as the catheter is initially empty, the operator may orient the saline-containing syringe orientation in any direction (e.g., pointing upward, downward, horizontally).

At block 1020, the operator injects a contrast (e.g., to confirm catheter location in the vasculature). Because the contrast is denser than saline, when injecting the contrast the operator orients the contrast-containing syringe to point the syringe upward.

At block 1030, the operator flushes the catheter with saline to flush out the contrast. Because saline is less dense than the contrast, the operator orients the saline-containing syringe to point the syringe downward.

At block 1035c, the operator fills the catheter with a pharmaceutically acceptable liquid that matches the density of DMSO. Because the DMSO (and therefore the pharmaceutically acceptable liquid) is denser than saline, the operator orients the liquid-containing syringe to point the syringe upward. One of ordinary skill in the art will be able to identify pharmaceutically acceptable liquids based on the metabolism of the biological subject and the known densities thereof compared to DMSO.

At block 1040c, the operator injects DMSO into the catheter in preparation for injecting the liquid embolic (e.g., per block 1050). Because DMSO is separated from the saline injected in block 1030 by the pharmaceutically acceptable liquid per block 1035c, the operator may orients the DMSO-containing syringe orientation to point the syringe in any direction, although most preferably downward to reduce the risk of introducing air bubbles.

At block 1050, the operator injects the liquid embolic (e.g., Onyx) to perform the procedure on the vasculature of the biological subject into the catheter. Because the liquid embolic is denser than the DMSO injected to block 1040c, the operator orients the embolic-containing syringe to point upward.

There are significant benefits to holding each liquid-filled syringe in a different direction based on the density (or specific gravity) of the liquid in the catheter and the liquid that will be injected from the filled syringes as described in FIGS. 10A-1C; orienting the syringe so that the more dense liquid is maintained in a lower orientation that the less dense liquid. Following the liquid flush/push sequence described in relation to FIGS. 10A-10C, the risk of premature solidification of liquid embolic or catheter rupture has been found to be reduced by up to 50%.

While the disclosure has been described in detail and with reference to specific illustrative embodiments thereof, it will be apparent to one skilled in the art that various changes and modifications can be made without departing from the spirit and scope of the disclosure. Accordingly, the present disclosure is not limited to that precisely as shown and described.

In addition to the embodiments described above, many examples of specific combinations are within the scope of the disclosure, some of which are detailed below:

Clause 1: A method, comprising: loading a first liquid having a first density into a vascular device hub having an internal volume flowably connected between a syringe port and a cannula port; connecting a first syringe loaded with a second liquid to the syringe port, wherein the second liquid has a second density different than the first density; orienting the first syringe at a first elevation relative to the vascular device hub to place a denser one of the first liquid and the second liquid below a less dense one of the first liquid and the second liquid; and injecting, from the first syringe into the vascular device hub, the second liquid to eject the first liquid from the vascular device hub via the cannula port.

Clause 2: The method as described in any of clauses 1 or 3-12, wherein the first liquid consists of a saline solution and the second liquid consists of an aqueous contrast agent solution.

Clause 3: The method as described in any of clauses 1-2 or 4-12, wherein the first liquid consists of an aqueous contrast agent solution, and the second liquid consists of a saline solution.

Clause 4: The method as described in any of clauses 1-3 or 5-12, wherein the first liquid consists of a saline solution, and the second liquid consists of a dimethyl sulfoxide solution.

Clause 5: The method as described in any of clauses 1-4 or 6-12, wherein the first liquid consists of a dimethyl sulfoxide solution, and the second liquid consists of a liquid embolic solution that is soluble in dimethyl sulfoxide but insoluble in aqueous solutions.

Clause 6: The method as described in any of clauses 1-5 or 7-12, further comprising: connecting a second syringe loaded with a third liquid to the syringe port, wherein the third liquid has a third density different than the second density; orienting the second syringe at a second elevation relative to the vascular device hub to place a denser one of the second liquid and the third liquid below a less dense one of the second liquid and the third liquid, wherein the second elevation is opposite to the first elevation; and injecting, from the second syringe into the vascular device hub, the third liquid to eject the second liquid from the vascular device hub via the cannula port.

Clause 7: The method as described in any of clauses 1-6 or 8-12, wherein when the second syringe is connected to the syringe port and oriented at a higher elevation than the vascular device hub, the third liquid is positioned above the second liquid, and the second liquid is positioned above the first liquid.

Clause 8: The method as described in any of clauses 1-7 or 9-12, wherein the first liquid consists of a saline solution, the second liquid consists of a dimethyl sulfoxide solution, and the third liquid comprises a liquid embolic soluble in dimethyl sulfoxide but insoluble in aqueous solutions.

Clause 9: The method as described in any of clauses 1-8 or 10-12, wherein the first syringe and the second syringe comprise longitudinal axes, and the longitudinal axes are not more than 45 degrees from vertical when injecting the second liquid and the third liquid, respectively.

Clause 10: The method as described in any of clauses 1-9 or 11-12, wherein the first syringe, when connecting to the vascular device hub, is oriented at an opposite orientation to a vertical reference axis than when the second liquid is injected into the vascular device hub.

Clause 11: The method as described in any of clauses 1-10 or 12, a volume of the second liquid is up to 500 microliters.

Clause 12: The method as described in any of clauses 1-11, further comprising: attaching (920) the cannula port to a blood vessel of a biological subject via a catheter; and wherein ejecting the first liquid via the cannula injects the first liquid into the blood vessel via the catheter.

Clause 13: A treatment process for arteriovenous malformations (AVMs) or aneurysms comprising the method of any of clauses 1-12.

Clause 14: The treatment process as described in clause 13, wherein the third liquid comprises a liquid embolic soluble in dimethyl sulfoxide but insoluble in aqueous solution that releases substantially all of the liquid embolic after injection of the third liquid into a target area in a biological subject.

Clause 15: A medical apparatus, comprising instructions for use, wherein the instructions for use comprise the method described in any one of clauses 1-12.

Certain terms are used throughout the description and claims to refer to particular features or components. As one skilled in the art will appreciate, different persons may refer to the same feature or component by different names. This document does not intend to distinguish between components or features that differ in name but not function.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims

1. A method (900), comprising:
loading (910) a first liquid (150a) having a first density into a vascular device hub (110) having an internal volume flowably connected between a syringe port (112) and a cannula port (114);
connecting (930) a first syringe (120) loaded with a second liquid (150b) to the syringe port, wherein the second liquid has a second density different than the first density;
orienting (940) the first syringe at a first elevation relative to the vascular device hub to place a denser one of the first liquid and the second liquid below a less dense one of the first liquid and the second liquid; and
injecting (950), from the first syringe into the vascular device hub, the second liquid to eject the first liquid from the vascular device hub via the cannula port.

2. The method of claim 1, wherein the first liquid consists of a saline solution and the second liquid consists of an aqueous contrast agent solution.

3. The method of claim 1, wherein the first liquid consists of an aqueous contrast agent solution, and the second liquid consists of a saline solution.

4. The method of claim 1, wherein the first liquid consists of a saline solution, and the second liquid consists of a dimethyl sulfoxide solution.

5. The method of claim 1, wherein the first liquid consists of a dimethyl sulfoxide solution, and the second liquid consists of a liquid embolic solution that is soluble in dimethyl sulfoxide but insoluble in aqueous solutions.

6. The method of claim 1, further comprising:
connecting (940) a second syringe (120) loaded with a third liquid (150c) to the syringe port, wherein the third liquid has a third density different than the second density;
orienting (940) the second syringe at a second elevation relative to the vascular device hub to place a denser one of the second liquid and the third liquid below a less dense one of the second liquid and the third liquid, wherein the second elevation is opposite to the first elevation; and
injecting (960), from the second syringe into the vascular device hub, the third liquid to eject the second liquid from the vascular device hub via the cannula port.

7. The method of claim 6, wherein when the second syringe is connected to the syringe port and oriented at a higher elevation than the vascular device hub, the third liquid is positioned above the second liquid, and the second liquid is positioned above the first liquid.

8. The method of claim 6, wherein the first liquid consists of a saline solution, the second liquid consists of a dimethyl sulfoxide solution, and the third liquid comprises a liquid embolic soluble in dimethyl sulfoxide but insoluble in aqueous solutions.

9. The method of claim 6, wherein the first syringe and the second syringe comprise longitudinal axes, and the longitudinal axes are not more than 45 degrees from vertical when injecting the second liquid and the third liquid, respectively.

10. The method of claim 1, wherein the first syringe, when connecting to the vascular device hub, is oriented at an opposite orientation to a vertical reference axis than when the second liquid is injected into the vascular device hub.

11. The method of claim 1, wherein a volume of the second liquid is up to 500 microliters.

12. The method of claim 1, further comprising:
attaching (920) the cannula port to a blood vessel of a biological subject via a catheter; and
wherein ejecting the first liquid via the cannula injects the first liquid into the blood vessel via the catheter.

13-15. (canceled)

16. The method of claim 1, wherein the second liquid floats on the first liquid and does not channel or mix with the first liquid.

17. A treatment process for arteriovenous malformations (AVMs) or aneurysms comprising the method of claim 1.

18. The treatment process of claim 17, wherein the third liquid comprises a liquid embolic soluble in dimethyl sulfoxide but insoluble in aqueous solution that releases substantially all of the liquid embolic after injection of the third liquid into a target area in a biological subject.

19. A medical apparatus, comprising instructions for use, wherein the instructions for use comprise the method of claim 1.

20. A prophylactic treatment process for arteriovenous malformations (AVMs) or aneurysms comprising the method of claim 1.

21. A method (900) of loading a single catheter and syringe assembly, the single catheter and syringe assembly comprising a vascular device hub (110) having an internal volume flowably connected between a syringe port (112) and a cannula port (114), the method comprising:
loading (910) a first liquid (150a) having a first density into the vascular device hub;
connecting (930) a first syringe (120) loaded with a second liquid (150b) to the syringe port, wherein the second liquid has a second density different than the first density;
orienting (940) the first syringe at a first elevation relative to the vascular device hub to place a denser one of the first liquid and the second liquid below a less dense one of the first liquid and the second liquid; and
injecting (950), from the first syringe into the vascular device hub, the second liquid to eject the first liquid from the vascular device hub via the cannula port.

22. The method of claim 21, wherein the second liquid floats on the first liquid and does not channel or mix with the first liquid.

23. The method of claim 21, wherein:
the first liquid consists of a saline solution and the second liquid consists of an aqueous contrast agent solution;
the first liquid consists of an aqueous contrast agent solution, and the second liquid consists of a saline solution;
the first liquid consists of a saline solution, and the second liquid consists of a dimethyl sulfoxide solution; or
the first liquid consists of a dimethyl sulfoxide solution, and the second liquid consists of a liquid embolic solution that is soluble in dimethyl sulfoxide but insoluble in aqueous solutions.
